(19) United States
(12) Patent Application Publication
(10) Pub. No.: US 2024/0425432 A1
(43) Pub. Date: Dec. 26, 2024

(54) Production and use of low or negative carbon eNaphtha

(71) Applicant: Infinium Technology, LLC, Sacramento, CA (US)

(72) Inventors: Robert Schuetzle, Sacramento, CA (US); Harold Wright, Menomonee Falls, WI (US); Benjamin Wright, Brookfield, WI (US)

(21) Appl. No.: 18/445,300

(22) Filed: Jun. 22, 2023

(51) Int. Cl. C07C 1/04; B01J 19/00; B01J 19/24; C01B 3/12; C07C 4/04; C07C 5/22; C25B 1/04

(52) U.S. Cl. CPC C07C 1/0485; B01J 19/0013; B01J 19/245; C01B 3/12; C07C 4/04; C07C 5/22; C25B 1/04; C01B 2203/0283; C01B 2203/062

(57) ABSTRACT

The present invention relates to a low-carbon eNaphtha. The low-carbon eNaphtha is produced through an electrofuel production process that utilizes a combination of CO2 and low carbon hydrogen. This invention addresses the need for environmentally friendly alternatives to traditional fossil fuel-based materials by providing a low-carbon eNaphtha solution. The disclosed low-carbon eNaphtha offers reduced greenhouse gas emissions and serves as a versatile resource for various applications. Its potential uses include but are not limited to fuel for transportation and energy generation, as well as a feedstock for the production of plastic precursors and other chemical compounds. The low-carbon eNaphtha represents a significant step towards achieving a sustainable and low-carbon future.

### FIELD OF THE INVENTION

The present invention relates to the novel production and utilization of eNaphtha, also known as electrofuel produced low carbon or negative carbon naphtha, for the purpose of plastic precursor synthesis. This invention particularly pertains to compositions comprising eNaphtha or materials derived from electrofuels produced from eNaphtha, or low carbon naphtha or negative carbon naphtha, which exhibit low carbon intensity and enable the efficient production of plastic precursors.

### BACKGROUND

Plastic is a ubiquitous material that is used in a variety of applications ranging from packaging to consumer products. It is a lightweight, durable, and inexpensive material that has become one of the most widely used materials in the world. However, the production of plastics is associated with a number of environmental impacts, including the release of carbon dioxide and other greenhouse gases.

The production of plastics begins with the selection of feedstocks. The most common feedstocks used today are petroleum, natural gas, natural gas liquids, and coal. To start the plastics production process, these feedstocks are broken down or “cracked” into their component molecules, which are then used as the starting materials for the plastics production process. The most commonly used starting materials for plastics production are ethylene, propylene, and butadiene. Ethylene and propylene are derived from natural gas or natural gas liquids or naphtha, while butadiene is typically derived from coal.

The next step in the production of plastics is the chemical transformation of the starting materials. This process involves the use of catalysts to break down the feedstocks into smaller molecules, which are then used to form the desired plastic product. The most common catalysts used are hydrofluoric acid, sulfuric acid, and phosphorus pentoxide. These catalysts are used to break down the feedstocks into molecules such as ethylene glycol, propylene glycol, and butadiene monomer. The final step in the production of plastics is the formation of the desired product. This is typically done by combining the molecules formed from the feedstocks with other chemicals, such as plasticizers, stabilizers, and colorants. The combination of these chemicals creates the desired plastic product.

The most carbon intensive step in the production of plastics is the production of the starting materials, such as ethylene, propylene, or butadiene, using what are referred to as naphtha or ethane “crackers”. Naphtha or ethane “crackers” are industrial plants that use a thermal cracking process to break down hydrocarbon molecules into smaller molecules. This process involves heating the feedstock (naphtha, ethane, or other feedstocks) to very high temperatures. Typically, the heating is done in a furnace at temperatures of around 1562° F. (850° C.) and then passing the heated materials through a catalyst bed, which causes them to separate into smaller molecules such as ethylene and propylene. The ethylene and propylene molecules are then cooled and collected for use. As discussed above, the ethylene and propylene are then further processed to create other chemicals such as plastics but may also be used to create detergents and synthetic fibers.

The carbon intensity of the naphtha or ethane cracker process is relatively high, as the process produces a significant amount of carbon dioxide (CO2) during the heating process for the cracker and the separation. The amount of CO2 produced depends on the type of hydrocarbon used and the efficiency of the process, but typically ranges from 1.5 to 2.5 tonnes of CO2 per tonne of ethylene produced.

According to the International Energy Agency, approximately 115 million metric tons of propylene is produced annually worldwide, and it is estimated that approximately 140 million metric tonnes of ethylene is produced annually around the world. If we assume that 2 tonnes of CO2 are produced per tonne of ethylene or propylene, then this equates to 510 million tonnes of CO2 annually from this production process a sizable amount of CO2.

Therefore, there is a need for the production of ethylene and propylene from alternative resources other than petroleum derived or coal derived products and using an alternate process other than the use of “crackers”.

A low-carbon or negative-carbon product like eNaphtha can be used to overcome a number of these problems. eNaphtha or electrofuel naphtha is produced from CO2. CO2 is captured and purified from a number of sources including biogenic sources (ex. ethanol, pulp, and paper, industrial (chemical plants, ammonia plants, other), from Direct Air Capture (removing CO2 from the atmosphere), or other types of CO2. H2 is produced by the electrolysis of water or other sources of low carbon hydrogen can be used including hydrogen produced from methane pyrolysis, geologic hydrogen, blue hydrogen from steam reforming using carbon capture, or other sources. The electrolysis used to create the “green hydrogen” is powered by low-carbon electricity including solar, wind, nuclear, hydro, biomass or derived other low carbon fuels.

### BRIEF SUMMARY OF THE INVENTION

The process for the production of low carbon intensity eNaphtha can impact the eNaphtha properties. Renewable or low carbon electricity is used to convert water to hydrogen and oxygen in an electrolyzer. Carbon dioxide is reacted with at least a portion of the hydrogen to produce a stream comprising carbon monoxide with a carbon dioxide conversion per pass of between 50% to 100%, preferably between 60% and 100%, and more preferably between 70% and 100%.

At least a portion of the carbon monoxide produced is reacted with hydrogen in a Liquid Fuel Production (LFP) reactor to produce a liquid product that comprises n-alkanes with carbon numbers from 4 to 24. The C₉ to C₁₅ fraction of the liquid product is between 40% and 100%, preferably between 45% and 100%, and more preferably between 50% and 100%. The LFP liquid product is further separated into three liquid streams in an LFP separation unit. The light LFP separation product stream comprises C₄ to C₈ n-alkanes; the medium LFP separation product stream comprises C₉-C₁₅ n-alkanes; the heavy LFP separation product stream comprises C₁₆-C₂₄ n-alkanes.

In some embodiments, at least a portion of the medium and/or heavy LFP separation unit product stream is fed to a catalytic hydroisomerization unit to produce a hydroisomerization product stream that is fed to a hydroisomerization separation unit to produce at least two hydroisomerization products wherein the heavy hydroisomerization product comprises branched chain alkanes with carbon numbers from 9 to 15 and wherein the freeze point of the heavy hydroisomerization product stream is −20° C. and even more preferably −40° C. or lower.

Optionally the full product stream may be sent to the hydro isomerization unit and then the hydroisomerized products are then fractionated. This configuration has the opportunity to upgrade the full product steam including some treatment of component in the eNaphtha stream, including small amounts of alcohols and/or olefins.

The eNaphtha as described herein is different than petroleum-based naphtha. The composition has significant differences including the fact that all the carbon in the naphtha is recycled carbon from other processes. The Carbon Intensity is low or negative because the CO₂ effectively has a very low CI via GREET and other methods. In one embodiment, the CI is less than 30 gCO₂/MJ. In another embodiment, the CI is less than 20 gCO₂/MJ. In yet another embodiment, the CI is less than 10 gCO₂/MJ. In another embodiment, the CI is 0 gCO₂/MJ or less (CI is negative).

### BRIEF DESCRIPTION OF THE DRAWING

FIG. 1 shows the process for the production of eNaphtha using low carbon electrolysis.

### DETAILED DESCRIPTION OF THE INVENTION

This invention relates to a unique production process for the production and use of eNaphtha in crackers to produce intermediaries for plastics production.

The specifications of the eNaphtha can vary depending on the plant design and operating conditions. In one embodiment, the eNaphtha contains nil sulfur and nil aromatics. Representative specifications are provided below in Table 1.

TABLE 1
eNaphtha Specifications

| Naphtha Specification | Naphtha |
|---|---|
| Fuel Energy Content (Btu/Gal) | 116,500 |
| Reid Vapor Pressure (psi) | 8.5 |
| Density (g/ml) (lbs/gal) (68°) | 0.680 (6.30) |
| Sulfur (ppm) | 0 |
| Aromatics (%) | 0 |
| Benzene (%) | 0 |
| Initial Boiling Point (IBP) | 84 |
| Olefin content | 0-20% |
| Alcohols/Hydroxy Alkanes | 0-5% |
| % of Iso-Paraffins | 5%-25% |
| % of Normal-Paraffins | 75%-95% |

Some properties of the eNaphtha are controllable including the olefin content, alcohols/hydroxy alkanes and the iso-paraffin vs. normal paraffin ratio and can be controlled by plant design and by varying operating conditions. Operating conditions include H2:CO ratio (higher H2:CO produces a lower olefin, alcohols, and iso-paraffin to normal ratios) and temperature, pressure. Other properties, including Reid Vapor Pressure, can be controlled by distillation or stabilization of the naphtha product at the plant. Specific desired properties of the eNaphtha can be produced based on the requirements of the cracker. For example, running the Liquid Fuel Production reactor at an H2:CO ratio greater than 2.0 can result in low to no olefins, alcohols/hydroxy-alkanes, and iso-paraffins.

The cracking yields, or the products that are produced from the eNaphtha in a cracker, of the eNaphtha are superior to petroleum derived or traditional naphtha.

TABLE 2
eNaphtha Cracking Yields

| | | Normal |
|---|---|---|
| | Infinium | Napththa |
| Ethylene | 35-40% | 25% |
| Propylene | 17-20% | 16% |
| Pygas | 12-14% | 22% |
| C4's | 8% | 12% |

As noted above when the eNaphtha is used, the production yields of ethylene and propylene are much improved while other undesirable products are reduced.

The electrofuel produced naphtha is a sustainable and renewable alternative to traditional fossil fuels. The use of this naphtha in the production of plastics reduces carbon emissions and mitigates the impact of plastics on the environment.

The technology disclosed in this patent application includes the use of electrofuel produced naphtha in the production of low carbon plastics. Naphtha is an organic compound derived from crude oil and is primarily used as a feedstock for petrochemicals production. The production of naphtha from crude oil is energy-intensive and contributes significantly to greenhouse gas emissions.

In contrast, electrofuel produced naphtha is derived from carbon dioxide captured from industrial processes such as power generation or cement production. This carbon dioxide is then combined with low carbon hydrogen, produced using renewable electricity and water. The electrofuel produced naphtha is a sustainable and renewable alternative to traditional naphtha derived from crude oil.

The electrofuel produced naphtha can be used in the production of a wide range of low carbon plastics. These plastics will have a significantly lower carbon footprint compared to traditional plastics. The low carbon plastics can be produced using a range of polymerization techniques such as injection molding, blow molding, and extrusion.

The properties of the low carbon plastics produced using electrofuel produced naphtha are similar to traditional plastics, however, do provide improved cracking yields as described above. These plastics have excellent mechanical, thermal, and chemical properties making them suitable for use in a wide range of applications. Since polypropylene and polyethylene based plastics are versatile materials they find use in a range of applications including packaging, automotive parts, household items, toys, medical equipment, and construction materials.

Packaging is one of the most important applications these plastics and they are widely used to produce various types of packaging such as bags, films, bottles, and containers. The reason for this widespread use is that these plastics offer outstanding barrier properties, meaning they can protect the contents of the package from external factors such as moisture, oxygen, and light. Additionally, these materials have excellent stability, which makes them suitable for use in frozen and refrigerated food packaging, as well as in packaging that requires high-temperature resistance.

Another area where this category of plastics are extensively used is in the automotive industry. Due to their light weight and high impact strength, these materials are ideal for producing automotive parts such as interior trims, bumpers, and dashboards. Furthermore, they offer excellent resistance to chemicals and UV radiation, which makes them suitable for use in exterior parts such as body panels and mirrors.

In addition to their use in packaging and automotive applications, this category of plastics are prevalent in household items and toys. These materials are widely used to produce household items such as hangers, buckets, and storage containers. They are also used to produce a variety of toys such as building blocks, puzzles, and dolls.

The medical sector is another important area in which these products are used. Due to their inertness, these materials are widely used in medical equipment such as syringes, IV bags, and blood collection tubes. Additionally, they are used to produce medical implants such as artificial joints and bone plates, due to their high strength, and biocompatibility.

Also commonly found in construction materials, these materials are used to produce a range of construction products such as pipes, insulation, and roofing materials. The reason for their widespread use in construction is that they are lightweight, durable, and have excellent chemical resistance.

Shoe manufacturers use both polyethylene and polypropylene plastics in the manufacturing process for different purposes. Polyethylene, in particular, is commonly used to make shoe soles and outsoles. The material's excellent shock absorption properties make it ideal for use in shoe soles. It also provides excellent grip, which offers better traction and stability when walking on different surfaces. Its flexibility allows it to conform to the contours of the feet, which makes it comfortable to wear. Additionally, polyethylene is extremely durable, which means it can withstand continuous wear and tear.

Polypropylene, on the other hand, is mostly used to make shoe uppers, the part of the shoe that covers the top of the foot. Polypropylene is often preferred because it is breathable and provides air circulation, making it ideal for use in shoes, especially in sports shoes where the feet get sweaty. It is also water-resistant, meaning it can protect the feet from moisture, making it useful for use in hiking boots and other outdoor shoes. Finally, polypropylene is highly durable, with strong resistance to abrasion, chemicals, and UV radiation, making it ideal for use in shoes that need to withstand various terrains.

The use of electrofuel produced naphtha in the production of low carbon plastics is a sustainable and renewable alternative to traditional fossil fuels. This technology reduces carbon emissions and mitigates the environmental impacts of plastics production. The low carbon plastics produced using electrofuel produced naphtha have a wide range of applications and can be used in various industries.

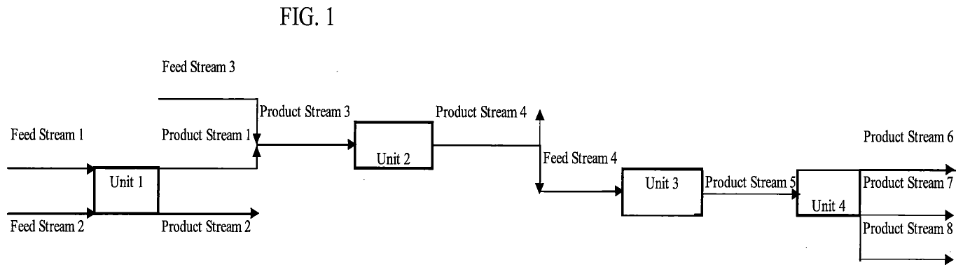

FIG. 1 shows an integrated process for the eNaphtha. Feed stream 1 is low carbon electricity. Low carbon power includes but is not limited to wind power, solar power nuclear power, power generated from biomass or renewable natural gas, and hydropower. Feed stream 2 is water. These are fed to Unit 1 which is the electrolyzer. In the electrolyzer, water and low carbon energy are used to produce hydrogen and oxygen. Water is fed to the electrolyzer. The electrolyzer is powered by low carbon electricity. Hydrogen is produced by electrolysis of water.

$${H}_{2} O = {H}_{2} + \frac{1}{2} {O}_{2}$$

Electrolyzers consist of an anode and a cathode separated by an electrolyte. Different electrolyzers function in slightly different ways. Different electrolyzer designs that use different electrolysis technology that are used include alkaline electrolysis, polymer electrolyte membrane (PEM) electrolysis, solid oxide electrolysis, high temperature electrolysis and other emerging types of electrolysis. Different electrolytes that are used including liquids KOH and NaOH, and with or without activating compounds. Activating compounds are added to the electrolyte to improve the stability of the electrolyte. Most ionic activators for the hydrogen evolution reaction are composed of an ethylenediamine-based metal chloride complex ([M(en)3]Clx,M¼Co, Ni, et al.) and Na₂MoO₄ or Na₂WO₄. Different electrocatalysts are used on the electrodes including many different combinations of metals and oxides like Raney-Nickel-Aluminum, which are enhanced by adding cobalt or molybdenum to the alloy.

The products from the electrolyzer are a stream comprising hydrogen called product stream 1 and a stream comprising oxygen called product stream 2. Due to the use of renewable energy sources, the electrolyzer produces green hydrogen. Depending on the carbon-intensity of energy used in unit 1 other low carbon hydrogen streams are produced. Other forms of hydrogen generation that may use renewable or non-renewable energy sources may also be used, including methane pyrolysis, steam reforming with carbon capture, biomass gasification, renewable natural gas (RNG) reforming or sourcing hydrogen from geological sources where purification of the stream may be required to produce hydrogen for use in a process.

In FIG. 1, Feed stream 3 is a stream that comprises carbon dioxide. Carbon dioxide is obtained from several sources. Power plants that generate electricity from various carbonaceous resources produce large amounts of carbon dioxide. Industrial manufacturing plants that produce ammonia for fertilizer produce large amounts of carbon dioxide. Ethanol plants that convert corn or wheat into ethanol produce large amounts of carbon dioxide via fermentation. Other industrial fermentation processes also produce large amounts of carbon dioxide. Municipal sewage treatment systems using aerobic and anaerobic digestion of sludge produce large amounts of CO₂. Utilization or conversion of CO₂, as described herein, typically involves separating and purifying the CO₂ from a gaseous stream where the CO₂ is not the major component (e.g., exhaust flue gas). Typically, an alkylamine is used to remove the carbon dioxide from the gas stream. Alkylamines used in the process include monoethanolamine, diethanolamine, methydiethanolamine, disopropylamine, aminoethoxyethnol, or combinations thereof. Metal Organic Framework (MOF) materials have also been used as a means of separating carbon dioxide from a dilute stream using chemisorption or physisorption to capture the carbon dioxide from the stream. Other methods to get concentrated carbon dioxide include chemical looping combustion where a circulating metal oxide material captures the carbon dioxide produced during the combustion process. Carbon dioxide is also captured from the atmosphere in what is called direct air capture (DAC). The processes for the capture of carbon dioxide often involve regeneration of the capture materials. Alkylamines are regenerated by being heated, typically by low pressure steam.

Captured carbon dioxide which is converted into useful products such as fuels (e.g. diesel fuel, gasoline blend stocks, jet fuel, other) and chemicals (e.g. solvents, olefins, alcohols, aromatics, other), displace fuels and chemicals produced from fossil sources such as petroleum and natural gas, lower the total net emissions of carbon dioxide into the atmosphere. This is what is meant by low carbon, very low carbon, zero carbon, or negative carbon fuels and chemicals.

The carbon dioxide streams that come from industrial or biological processes, or is captured from the atmosphere, or that is available from a commercial carbon dioxide pipeline is not pure carbon dioxide. These available carbon dioxide streams from industrial facilities or pipelines contain sulfur containing compounds from none to 2000 parts per million by weight and also contain hydrocarbons from none to 10 volume percent. Purification of the carbon dioxide including the removal of sulfur containing compounds and hydrocarbons is necessary to avoid issues with downstream processing. After purification, the purified carbon dioxide is suitable for the generation of low carbon or zero-carbon fuels and chemicals.

At least a portion of the product stream 1 comprising hydrogen is blended with the feed stream 3 comprising carbon dioxide to produce a RWGS Reactor feed stream, Product Stream 3. Carbon dioxide and hydrogen are reacted to carbon monoxide and water in a Reverse Water Gas Shift (RWGS) reactor where the heat of reaction is provided by a RWGS Heater. The catalyst used in the RWGS reactor is a catalyst as referenced in the published application U.S. Ser. No. 17/300,260.

At least a portion of Product stream 4 (RWGS Product stream) which comprises hydrogen and carbon monoxide becomes the Liquid Fuel Production (LFP) reactor feed stream, Feed Stream 4. The RWGS Product, comprising carbon monoxide and with the possible addition of extra hydrogen, is reacted to fuels and chemicals in a Liquid Fuels Production (LFP) reactor that uses a catalyst to produce long chain hydrocarbons that are used as fuels and chemicals. The final product is a hydrocarbon mixture where the majority (e.g., 51 volume percent to 99 volume percent) of hydrocarbons in the mixture are hydrocarbons 4 to 24 carbon atoms in length.

At least a portion of the LFP hydrocarbon product stream (Product Stream 5) comprises n-alkanes with carbon numbers from 4 to 24 that is fed to an LFP separation unit where at least three products are produced. LFP separation is any separation process absorption, adsorption, filtration, or distillation. Distillation is the preferred separation process. The LFP separation unit produces at least three products. The light LFP separation product, Product Stream 8, comprises n-alkanes with carbon numbers between 4 and 8. The heavy LFP separation product, Product Stream 6, comprises n-alkanes with carbon numbers between 16 and 24. The medium LFP separation product, Product Stream 7, comprises n-alkanes from carbon numbers between 9 and 15.

In one embodiment of the invention, at least a portion of the heavy LFP separation unit product is sold as premium low sulfur, high cetane diesel fuel.

In some embodiments, at least a portion of the medium LFP separation product is fed to a hydroisomerization unit. In hydroisomerization, the properties of the feedstock are improved by transforming normal alkane hydrocarbons to branched ones having the same carbon numbers. This reaction improves the cold flow properties of the hydrocarbon.

In some embodiments, all the fractions of the LFP are fed to a hydroisomerization unit either after distillation or before the distillation unit. This approach can improve not only the qualities of the distillate fuels but also can tailor the properties of the eNaphtha to increase the percentage of n-paraffins in the eNaphtha.

In some embodiments, only the light LFP separation product is fed to a hydroisomerization unit in order to improve the properties of the eNaphtha to increase the percentage of n-paraffins in the eNaphtha.

The hydroisomerization reactor is any suitable design but it is preferred that the reactor be a cylindrical reactor with a liquid feed at the top of the reactor. The liquid feed comprising the medium LFP separation product is mixed with hydrogen. The combined feed reacts over a catalyst bed in the reactor vessel. Typically, this reactor is a trickle bed reactor. At least a portion of the n-alkanes react across the catalyst to produce branched alkanes. The reactor is comprised of one or more catalyst beds. In some embodiments, additional hydrogen feed is injected between the various catalyst beds. The molar ratio of the hydrogen to liquid hydrocarbon feed ranges from 10 to 300, more preferably between 15 to 30, even more preferably from 19 to 25. The operating pressure of the hydroisomerization reactor is between 10 to 100 bar, more preferably between 20 and 80, and more preferably between 30 and 40. The Weight Hourly Space Velocity (WHSV) is between 0.1 to 10 kg/hr liquid feed/kg catalyst, more preferably between 0.2 and 5 hr⁻¹, and more preferably between 0.5 to 2 hr⁻¹. The reactor operating temperature from 200° C. to 350° C.

The hydroisomerization catalyst is a solid shaped particle. The catalyst comprises a metal deposited on an acidic support. The catalyst metal in some embodiments is a platinum and palladium that provides hydrogenation and dehydrogenation activity. The catalyst metal in some embodiments comprises nickel. The catalyst metal in some embodiments comprises copper. In some embodiments that catalyst metal comprises a bimetallic such as Ni—Cu, Ni—Mo, Pt—Fe, and Pt—Be. The acidic support is chosen from any suitable support and includes supports comprising ZSM-5, ZSM-22, ZSM-23, Silica, Alumina, SiO₂—Al₂O₃, Beta zeolite, MCM-41, MCM-48, SBA-15 and includes blends of such supports.

The conversion of n-alkane to branched alkane in the hydroisomerization reactor is preferably between 50 and 100%, and more preferably from 80 to 100%. The reactor temperature, pressure, hydrogen to n-alkane ratio, and weight hourly space velocity (WHSV) is manipulated to maintain a high conversion of n-alkane conversion to branched alkanes.

In one embodiment, the specifications of the eNaphtha can vary depending on the plant design and operating conditions. The eNaphtha contains less than 1 ppm sulfur and less than 0.1 wt % aromatics. Representative specifications are shown above in Table 1

As can be seen from Table 2, the naphtha produced from recycled CO2 can exhibit results that are very different from traditional naphtha. The eNaphtha has lower sulfur, and almost no aromatics and higher n-paraffins. The embodiment eNaphtha has less than 5 ppm sulfur. In another embodiment, the sulfur is less than 1 pm.

Petroleum naphtha is often processed in a naphtha reformer. The mechanism of naphtha reforming over conventional Pt-based chlorinated alumina catalysts requires a rich naphtha with high N+2A (N-paraffin+2×aromatics) ratio is required a product with high RON without too much liquid yield loss. Therefore, naphtha reforming is not the reforming technology of choice for eNaphtha. Steam cracking is the preferred naphtha conversion technology for this eNaphtha. In another embodiment, the process for producing propylene and ethylene through eNaphtha steam cracking. The cracking yields of the eNaphtha are superior to petroleum derived or traditional naphtha as shown in Table 2.

In one embodiment, a low-carbon propylene and ethene and low carbon polypropylene and polyethylene are produced. These materials are made from CO2.

A polymerization reaction starts with a primary ingredient (monomer), such as ethylene or propylene. Ethylene (C₂H₄) is a stable molecule with two carbon atoms and a double bond. Polyethylene (PE) is a made by the reaction of multiple ethylene molecules in the presence of catalyst to break the double bond and connect the carbon atoms into a chain. The longer the chain, the higher the molecular weight. Polymers can have molecular weights in the millions. Similarly, polypropylene (PP) is made by breaking the double bond in a propylene (C₃H₆) molecule, in the presence of a catalyst, to form long chains of three-carbon-atom molecules. The third carbon atom adds a complexity: The methyl (CH₃) groups fall on one side of the chain's centerline, or backbone (isotactic), or they could appear alternately on opposite sides of the backbone (syndiotactic), or their positions could be random (atactic). These arrangements have different physical properties.

Polymerization reactions also consume hydrogen, which is required to quench the reaction (i.e., end the chains), and some will involve a secondary ingredient (known as a comonomer). In one embodiment, the hydrogen is produced from the electrolysis system. This means that these polymers are made from CO2 and do not use fossil fuels.

Since the concentrations of these components in the reactor affect the probabilities that specific reactions will take place, the composition in the polymer reactor effectively sets the amount of branching and the length of the chain.

PE and PP processes require very pure monomer and comonomer(s) to minimize undesirable side reactions that might affect the polymer structure and properties. Most ethylene and propylene producers make both chemical and polymer grades. Typical polymer-grade ethylene and propylene are at least 99.95% pure, with limits on certain impurities (e.g., acetylene and propidine) in the parts-per-million range. Depending on the feed source, some polymer plants require one or more distillation columns at the front end to purify the raw material or to recycle the unreacted monomers to the reactors.

1. A process for producing ethylene and propylene from eNaphtha, wherein the process comprises:
a) feeding low carbon electricity and water to an electrolyzer to produce a first stream comprising hydrogen and a second stream comprising oxygen;
b) blending at least a portion of the first stream with a third stream comprising carbon dioxide to produce a fourth stream, wherein the fourth stream is fed into a Reverse Water Gas Shift Reactor to provide a fifth stream comprising carbon monoxide and hydrogen;
c) feeding at least a portion of the fifth stream to a Liquid Fuels Production reactor, wherein the Liquid Fuels Production reactor comprises a catalyst, to provide a sixth stream which is eNaphtha comprising a hydrocarbon mixture, wherein between 51 volume percent and 99 volume percent of the sixth stream are hydrocarbons 4 to 24 carbon atoms in length;
d) feeding at least a portion of the sixth stream to a Liquid Fuels Production separation unit to produce a seventh stream comprising n-alkanes having carbon numbers between 4 and 8, an eighth stream comprising n-alkanes having carbon numbers between 9 and 15 and a ninth stream comprising n-alkanes having carbon numbers between 16 and 24;
e) feeding at least one of seventh, eighth or ninth streams to a hydroisomerization reactor to produce a tenth stream;
f) subjecting the tenth stream to steam cracking thereby producing and eleventh stream comprising ethylene and propylene, wherein the ethylene comprises from 26 volume percent to 45 volume percent of the eleventh product stream and propylene comprises from 17 volume percent to 25 volume percent of the eleventh stream.

2. The process according to claim 1, wherein the eNaphtha has a Carbon Intensity ranging from 0 gCO₂/MJ and 40 gCO₂/MJ.

3. The process according to claim 1, wherein the hydroisomerization reactor operates at a pressure ranging from 10 bar to 100 bar.

4. The process according to claim 1, wherein the eNaphtha contains between 0 ppm and 1 ppm sulfur and between 0 wt % and 0.1 wt % aromatics.

5. The process according to claim 1, wherein the Weight Hourly Space Velocity in the hydroisomerization reactor ranges from 0.1 to 10 kg/hr liquid feed/kg catalyst.

6. The process according to claim 2, wherein the eNaphtha has a Carbon Intensity ranging from 0 gCO₂/MJ and 40 gCO₂/MJ, and wherein the hydroisomerization reactor operates at a pressure ranging from 10 bar to 100 bar, and wherein the eNaphtha contains between 0 ppm and 1 ppm sulfur and between 0 wt % and 0.1 wt % aromatics, and wherein the Weight Hourly Space Velocity n the hydroisomerization reactor ranges from 0.1 to 10 kg/hr liquid feed/kg catalyst.

7. A process for making a low-carbon polymer from eNaphtha, wherein the low-carbon polymer is either low-carbon polyethylene or low-carbon polypropylene, wherein the process comprises:
a) feeding low carbon electricity and water to an electrolyzer to produce a first stream comprising hydrogen and a second stream comprising oxygen;
b) blending at least a portion of the first stream with a third stream comprising carbon dioxide to produce a fourth stream, wherein the fourth stream is fed into a Reverse Water Gas Shift Reactor to provide a fifth stream comprising carbon monoxide and hydrogen;
c) feeding at least a portion of the fifth stream to a Liquid Fuels Production reactor, wherein the Liquid Fuels Production reactor comprises a catalyst, to provide a sixth stream which is eNaphtha comprising a hydrocarbon mixture, wherein between 51 volume percent and 99 volume percent of the sixth stream are hydrocarbons 4 to 24 carbon atoms in length;
d) feeding at least a portion of the sixth stream to a Liquid Fuels Production separation unit to produce a seventh stream comprising n-alkanes having carbon numbers between 4 and 8, an eighth stream comprising n-alkanes having carbon numbers between 9 and 15 and a ninth stream comprising n-alkanes having carbon numbers between 16 and 24;
e) feeding at least one of seventh, eighth or ninth streams to a hydroisomerization reactor to produce a tenth stream;
f) subjecting the tenth stream to steam cracking, thereby producing and eleventh stream comprising ethylene and propylene, wherein the ethylene comprises from 26 volume percent to 45 volume percent of the eleventh product stream and propylene comprises from 17 volume percent to 25 volume percent of the eleventh stream;
g) separating the ethylene and propylene to provide purified ethylene and propylene;
h) subjecting either the purified ethylene or propylene to a polymerization reaction, thereby producing low-carbon polyethylene or low-carbon polypropylene.

8. The process for making a low-carbon polymer according to claim 7, wherein the eNaphtha has a Carbon Intensity ranging from 0 gCO₂/MJ and 40 gCO₂/MJ.

9. The process for making a low-carbon polymer according to claim 7, wherein the hydroisomerization reactor operates at a pressure ranging from 10 bar to 100 bar.

10. The process for making a low-carbon polymer according to claim 7, wherein the eNaphtha contains between 0 ppm and 1 ppm sulfur and between 0 wt % and 0.1 wt % aromatics.

11. The process for making a low-carbon polymer according to claim 7, wherein the Weight Hourly Space Velocity in the hydroisomerization reactor ranges from 0.1 to 10 kg/hr liquid feed/kg catalyst.

12. The process for making a low-carbon polymer according to claim 8, wherein the eNaphtha has a Carbon Intensity ranging from 0 gCO₂/MJ and 40 gCO₂/MJ, and wherein the hydroisomerization reactor operates at a pressure ranging from 10 bar to 100 bar, and wherein the eNaphtha contains between 0 ppm and 1 ppm sulfur and between 0 wt % and 0.1 wt % aromatics, and wherein the Weight Hourly Space Velocity n the hydroisomerization reactor ranges from 0.1 to 10 kg/hr liquid feed/kg catalyst.

13. A system for producing ethylene and propylene from eNaphtha, wherein the system comprises:
a) an electrolyzer for producing hydrogen and oxygen from water;
b) a Reverse Water Gas Shift Reactor for producing carbon monoxide and hydrogen from hydrogen and carbon dioxide;
c) a Liquid Fuels Production reactor for producing eNaphtha comprising a hydrocarbon mixture, from carbon monoxide and hydrogen;
d) a Liquid Fuels Production separation unit for separating the hydrocarbon mixture into different fractions;
e) a hydroisomerization reactor for converting normal alkanes in the hydrocarbon mixture to branched alkanes;
f) a steam cracking unit for converting branched alkanes into ethylene and propylene.

14. The system according to claim 13, wherein the electrolyzer uses polymer membrane electrolysis or solid oxide electrolysis technology.

15. The system according to claim 13, wherein there is a heat of reaction for the Reverse Water Gas Shift Reactor, and wherein the heat of reaction is provided by a Reverse Water Gas Shift Heater.
